(19) United States
(12) Patent Application Publication
(10) Pub. No.: US 2014/0276069 A1
(43) Pub. Date: Sep. 18, 2014

(54) ULTRASOUND PROBE

(71) Applicant: EagIEyeMed, Santa Clara, CA (US)

(72) Inventors: Ravi Amble, San Jose, CA (US); Farooq Raza, Milpitas, CA (US)

(73) Assignee: EagIEyeMed, Santa Clara, CA (US)

(21) Appl. No.: 14/214,370

(22) Filed: Mar. 14, 2014

Related U.S. Application Data
(60) Provisional application No. 61/800,437, filed on Mar. 15, 2013.

(51) Int. Cl. A61B 8/08; A61B 19/00; A61B 8/14; A61B 8/00

(52) U.S. Cl. CPC A61B 8/5207; A61B 8/4488; A61B 8/4455; A61B 8/56; A61B 8/546; A61B 19/5202; A61B 8/145; A61B 8/4472; A61B 8/4411
USPC 600/447

(57) ABSTRACT

In one general embodiment, an ultrasound probe includes a housing configured for grasping by a human hand; an array of transducers for transducing sound waves into electrical signals; a circuit board in the housing, the circuit board having a plurality of leads, each of the transducers being coupled to at least an associated one of the leads; processing circuitry in the housing and coupled to the circuit board for processing the electrical signals, or derivatives of the electrical signals, into sonogram data; and an output device for outputting the sonogram data.

### RELATED APPLICATIONS

This application claims priority from U.S. Provisional Patent Application No. 61/800,437 filed on Mar. 15, 2013, which is herein incorporated by reference.

### FIELD OF THE INVENTION

The present invention relates to ultrasound technology, and more particularly, this invention relates to a handheld ultrasound probe that performs most or all ultrasound processing internally.

### BACKGROUND

Ultrasound machines emit and receive sound waves which are used to generate an image of objects positioned in the field of the sound waves. Specifically, a transducer probe generates sound waves that are then propagated outward. Moreover, the transducer probe is able to detect reflected sound waves.

Transducer probes of conventional ultrasound machines are connected to a remote central processing unit using a relatively long (about 2-m) cable which may contain anywhere from 48 to 256 micro-coaxial cables. However, the micro-coaxial cables are very expensive and account for a significant amount of the cost of manufacturing conventional ultrasound machines.

Additionally, the 48 to 256 micro-coaxial cables are bundled together to form a thicker connection that is heavy and difficult to manipulate. As a result, the bundled cables cause users of such conventional ultrasound machines to become overburdened thereby causing severe pain and fatigue while trying to use the machine. Attempting to capture the best ultrasound image takes a trained sonographer several attempts to get the correct angle and cover the region of interest. Thus, the bulky and burdensome conventional ultrasound machines unnecessarily burden users.

Higher frequencies are in principle more desirable, since they provide higher resolution. However, tissue attenuation limits how high the frequency can be for a given penetration distance. Thus, it is not desirable that the ultrasound frequency be arbitrarily increased to get improved resolution, as the corresponding signal experiences an attenuation of about 1 dB/cm MHz. According to an example, for a 10-MHz ultrasound signal and a penetration depth of 5 cm, the round-trip signal has been attenuated by 5×2×10=100 dB. Thus, in order to accommodate an instantaneous dynamic range of about 60 dB at any location, the required dynamic range would be about 160 dB which may correspond to a voltage dynamic range of 100 million to 1.

Dynamic ranges of this magnitude may not be directly achievable conventionally. Rather, conventional products, although highly sophisticated system, have limited penetration depth (e.g., limited by safety regulations due to maximum transmit power that is allowed) and/or image resolution (e.g., using a lower ultrasound frequency).

Furthermore, cable mismatch and cable losses of the micro-coaxial cables directly contribute to the noise figure (NF) of conventional ultrasound machines as a whole. For example, if the loss of the cable at a particular frequency is 2 dB, then the NF is degraded by 2 dB. As a result, the first amplifier after the cable will have to have a noise figure that is 2 dB lower than would be associated with a lossless cable.

Moreover, as the operational frequency of the transducers in the transducer probe increases, the wavelength and consequently the performance area decrease, thereby resulting in an increased element impedance (i.e., a reduced capacitance value causes an increase to the real part of the impedance). Furthermore, increased transducer element impedances have the strong disadvantage that it becomes ever more difficult to drive the cable directly. For example, a conventional 2 m cable might have a capacitance of 203 pF, while a transducer element could have capacitance on the order of 5 pF. This undesirably makes for a large capacitive attenuator in conventional ultrasound machines.

### SUMMARY

An ultrasound probe according to one embodiment includes a housing configured for grasping by a human hand; an array of transducers for transducing sound waves into electrical signals; a circuit board in the housing, the circuit board having a plurality of leads, each of the transducers being coupled to at least an associated one of the leads; processing circuitry in the housing and coupled to the circuit board for processing the electrical signals, or derivatives of the electrical signals, into sonogram data; and an output device for outputting the sonogram data.

An ultrasound probe according to one embodiment includes a housing configured for grasping by a human hand; an array of piezoelectric transducers for generating sound waves and for transducing reflected ones of the sound waves into electrical signals; a flexible circuit board in the housing, the circuit board having a plurality of leads, each of the transducers being coupled to at least an associated one of the leads; processing circuitry in the housing and coupled to the circuit board for processing the electrical signals, or derivatives of the electrical signals, into sonogram data; an output device for outputting the sonogram data, wherein the output device includes a wireless transmitter; a battery for powering the processing circuitry; and a control on the housing for adjusting at least one of a frequency and depth of the sound waves.

Other aspects and advantages of the present invention will become apparent from the following detailed description, which, when taken in conjunction with the drawings, illustrate by way of example the principles of the invention.

### BRIEF DESCRIPTION OF THE DRAWINGS

For a fuller understanding of the nature and advantages of the present invention, as well as the preferred mode of use, reference should be made to the following detailed description read in conjunction with the accompanying drawings.

FIG. 1A is a representational diagram of an ultrasound system, according to one embodiment.

FIG. 1B is a representational diagram of an ultrasound system, according to one embodiment.

FIG. 2A is a representational diagram of an ultrasound system having a wireless ultrasound probe, according to one embodiment.

FIG. 2B is a detailed view of a section of the wireless ultrasound probe of FIG. 2A.

FIG. 2C is a kit, according to one embodiment.

FIG. 3 is a single element piezoelectric transducer according to one embodiment.

FIG. 4 is a representational diagram of an analog front end (AFE) according to one embodiment.

FIG. 5 is a representational diagram of the digital front end according to one embodiment.

FIG. 6 is a representational diagram of a digital back end processing engine according to one embodiment.

FIG. 7 is an ultrasonic probe according to one embodiment.

FIG. 8 is a linear array contact head according to one embodiment.

FIG. 9 is a representational diagram of a transducer component array according to one embodiment.

FIG. 10 is a micro-convex contact head according to one embodiment.

FIG. 11 is a phased array transducer contact head according to one embodiment.

FIG. 12 is a partial view of a battery compartment having a battery according to one embodiment.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified. Furthermore, as used herein, the term “about” with reference to some stated value refers to the stated value ±10% of said value.

The following description discloses several preferred embodiments of ultrasound probes and/or related systems and methods.

In one general embodiment, an ultrasound probe includes a housing configured for grasping by a human hand; an array of transducers for transducing sound waves into electrical signals; a circuit board in the housing, the circuit board having a plurality of leads, each of the transducers being coupled to at least an associated one of the leads; processing circuitry in the housing and coupled to the circuit board for processing the electrical signals, or derivatives of the electrical signals, into sonogram data; and an output device for outputting the sonogram data.

In another general embodiment, an ultrasound probe includes a housing configured for grasping by a human hand; an array of piezoelectric transducers for generating sound waves and for transducing reflected ones of the sound waves into electrical signals; a flexible circuit board in the housing, the circuit board having a plurality of leads, each of the transducers being coupled to at least an associated one of the leads; processing circuitry in the housing and coupled to the circuit board for processing the electrical signals, or derivatives of the electrical signals, into sonogram data; an output device for outputting the sonogram data, wherein the output device includes a wireless transmitter; a battery for powering the processing circuitry; and a control on the housing for adjusting at least one of a frequency and depth of the sound waves.

As previously mentioned, transducer probes of conventional ultrasound machines are connected to a central processing unit using a relatively long (about 2-m) cable which may contain anywhere from 48 to 256 micro-coaxial cables. These micro-coaxial cables are very expensive and account for a significant amount of the cost of conventional ultrasound machines. Furthermore, cable mismatch and cable losses of the micro-coaxial cables directly contribute to the noise figure (NF) of conventional ultrasound machines as a whole.

Moreover, as the operational frequency of the transducers in the transducer probe increases, the wavelength and consequently the performance area decrease, thereby resulting in increased element impedance. Increased transducer element impedances have the strong disadvantage that it becomes ever more difficult to drive the cable directly. This undesirably makes for a large capacitive attenuator in conventional ultrasound machines.

In sharp contrast, various embodiments described herein include handheld, ultrasound probes that overcome the foregoing limitations of conventional products. As will be described in further detail below, various embodiments described below introduce using flexible printed circuits boards, thereby obviating the need for micro-coaxial cables. Therefore, various approaches herein increase functionality while reducing user encumbrance, as well as cost by not having micro-coaxial cables extending between a probe and a backend processing system.

An ultrasound examination, also known as “ultra-sonography”, is a non-invasive imaging technique that allows internal body structures to be seen by recording echoes or reflections of ultrasonic waves. Unlike X-rays, which are potentially dangerous, ultrasound waves are relatively safe.

Ultrasound equipment directs a narrow beam of high frequency sound waves from 2 MHz to 20 MHz into an area of interest, e.g., of a patient. The sound waves may be transmitted through, reflected or absorbed by the tissues that they encounter. The ultrasound waves that are reflected will return as “echoes” to the probe and these echoes are then converted into an image by the computing device that the probe it is connected to.

Looking to FIG. 1A, an exemplary ultrasound system 100 is illustrated according to one embodiment. Although FIG. 1A depicts an exemplary embodiment, as an option, the present ultrasound system 100 may be implemented in conjunction with features from any other embodiment listed herein, such as those described with reference to the other FIGS., such as FIG. 1B. Of course, however, such ultrasound system 100 and others presented herein may be used in various applications and/or in permutations which may or may not be specifically described in the illustrative embodiments listed herein. Further, the ultrasound system 100 presented herein may be used in any desired environment. Thus FIG. 1A (and the other FIGS.) should be deemed to include any and all possible permutations.

As illustrated, the ultrasound system 100 includes an ultrasound probe 122 that is connected to an AFE 124, beamformer and digital front end 126 and digital back end and display 128. As will be described in further detail below, the ultrasound probe 122 is preferably wirelessly connected to the AFE 124 which may be positioned in a housing (e.g., see 101 of FIG. 1B).

Depending on the mode of operation for the system 100, the digital back end and display 128 may show a 2-dimensional “picture” of the tissues and/or organs that are under examination. The technique is invaluable for the examination of internal organs in both veterinary medicine and human medicine, e.g., for pregnancy diagnosis. Other in-use embodiments may include evaluating heart conditions and/or identifying changes in abdominal organs of a patient. Furthermore, Ultra-sonography is useful in the diagnosis of cysts, tumors and other similar growths.

The basic functionality of an ultrasound system (e.g., machine) includes using transducers to focus sound waves along the scan lines in a region of interest, e.g., of a human patient. The term ultrasound refers to frequencies that are greater than about 20 kHz, which is commonly accepted to be the upper frequency limit the human ear can hear. Typically, ultrasound systems operate in the 2 MHz to 20 MHz frequency range, although some systems may approach 40 MHz or higher, e.g., for harmonic imaging.

It should also be noted that the number of sound waves emitted determines the resulting field of view, while the frequency of the emitted sound waves determines the resolution of the resulting image generated from the reflected sound waves and the power of the emitted sound waves determines how deep the sound waves penetrate. According to the present description, the desired depth to which the sound waves penetrate may vary depending on the in-use application. For example, ultrasonic imaging of the biological construction of a knee may use a shallower depth of sound wave penetration than an instance developing ultrasonic imaging of an internal organ, e.g., a heart.

Looking to FIG. 1B, an exemplary ultrasound system 150 is illustrated, in accordance with one embodiment. Although FIG. 1B depicts an exemplary embodiment, as an option, the present ultrasound system 150 may be implemented in conjunction with features from any other embodiment listed herein, such as those described with reference to the other FIGS. Of course, however, such ultrasound system 150 and others presented herein may be used in various applications and/or in permutations which may or may not be specifically described in the illustrative embodiments listed herein. Further, the ultrasound system 150 presented herein may be used in any desired environment. Thus FIG. 1B (and the other FIGS.) should be deemed to include any and all possible permutations.

Referring now to FIG. 1B, the ultrasound system 150 includes a housing 101 in which a processing component 102 is positioned having a beamformer control unit 104. The beamformer control unit 104 preferably synchronizes the generation of the sound waves and the reflected sound wave measurements detected, as will soon become apparent.

The beamformer control unit 104 translates the desired resulting image, e.g., in terms of field of view and depth, into a corresponding number of scan lines and focal points per scan line. According to one approach, the beamformer control unit 104 may begin with a first of the scan lines and thereby excite an array of piezoelectric transducers (e.g., see 110) with a sequence of high voltage pulses via transmit amplifiers. Illustrative operational characteristics for the amplifiers may be about 100 V and about 2 Amps for each piezoelectric transducer, but could be higher or lower, depending on the desired embodiment.

Ultrasound system 150 further includes a digital to analog converter 106, e.g., of a type known in the art, in addition to a transmit/receive (T/R) switch 108. The digital to analog converter 106 preferably converts the voltage pulses to an analog signal as would be understood by one skilled in the art upon reading the present description. Moreover, the T/R switch 108 preferably prevents the high voltage pulses from damaging the circuitry and/or electronics of the system 150.

The transducers 110, which are preferably piezoelectric transducers, are positioned towards an edge of the system 150, preferably the housing 101. Thus, the transducers 110 may be able to emit uninterrupted sound waves corresponding to the high voltage pulses received. As will be described in further detail below, additional components of the system 150 are also preferably positioned in and/or on a wireless ultrasound probe. Thus, in some embodiments system 150 may represent an exemplary embodiment of a wireless ultrasound probe.

Referring still to FIG. 1B, according to some approaches, the high voltage pulses may be properly time delayed so that the resulting sound waves may be focused along a desired scan line. By focusing the sound waves along a desired scan line, the system 150 may be able to produce a narrowly focused beam at a desired focal point. According to one approach, the beamformer control unit 104 may determine which of the transducers 110 to energize at a given time and/or the proper time delay to apply for each of the transducers 110 to properly steer the sound waves towards the desired focal point.

As sound waves propagate toward a desired focal point, the sound waves migrate through materials with different densities. Upon experiencing each change in density, the sound wave may change direction slightly, and also creates a reflected sound wave. Some of the reflected sound waves propagate back to the transducers 110 and the reflected sound waves are thereby detected and form a reflected input signal for the system 150.

However, reflected input signals usually have weak amplitudes, thereby resulting in low voltage signals in the transducers 110. Moreover, low voltage signals can cause increased noise, decreased signal accuracy, etc. Thus, in some embodiments the system 150 may use one or more variable controlled amplifiers (VCAs) 112 to scale the low voltage reflected input signals.

The VCA 112 is preferably used before the input signal is sampled by an analog-to-digital converter (ADC) 114. In one approach, the VCA may be configured such that the gain profile being applied to the reflected input signal is a function of the sample time in view of the fact that the signal strength decreases with time (e.g., it has traveled through more tissue). Furthermore, in various embodiments the ADC sampling rate may be 4 or more times higher than the transducer center frequency.

As mentioned above, the system 150 may use one or more VCAs 112. It follows that depending on the desired embodiment, the number of VCA and ADC combinations may be determined by the number of transducers 110 included in the system 150.

Referring still to FIG. 1B, once the input signals reach the processing component 102, the signals may be scaled and/or appropriately delayed, e.g., by the beamformer control unit 104, to permit a coherent summation of the signals. Once the input signals are scaled and/or appropriately delayed, each of these updated signals may correspond to one or more of the focal points along a particular scan line. Any such processing of the input signals may be performed in an application-specific integrated circuit (ASIC), a field-programmable gate array (FPGA), a digital signal processing (DSP) unit, etc. and/or a combination thereof. The device(s) used to perform the processing may depend on the number of transducers used in a particular embodiment, which determines the input/output (I/O) requirement as well as the processing requirement of the associated embodiment.

In some approaches, a high voltage multiplexer and/or demultiplexer (not shown) may be used in some arrays, e.g., to reduce the complexity of transmit and receive hardware of some embodiments. Moreover, in other approaches, phased-array digital beamformer systems may be used, as would be appreciated by one skilled in the art upon reading the present description.

Components of the system 150 and others described herein may be constructed using conventional techniques and designs, and may be adapted for use in such embodiments, as would become apparent to one skilled in the art only upon reading the present description.

As mentioned above, one or more components from the system 150 of FIG. 1B may be implemented in a wireless ultrasound probe. A wireless ultrasound probe is desired as it overcomes the limitations of conventional ultrasonic machines having micro-coaxial cables, which increase initial costs and upkeep costs, in addition to increasing the losses experienced by the signal being transferred through the cables.

FIGS. 2A-2B depict a representational diagram of an ultrasound system 200 having a wireless ultrasound probe 203, in accordance with one embodiment. As an option, the present ultrasound system 200 may be implemented in conjunction with features from any other embodiment listed herein, such as those described with reference to the other FIGS. Of course, however, such ultrasound system 200 and others presented herein may be used in various applications and/or in permutations which may or may not be specifically described in the illustrative embodiments listed herein. Further, the ultrasound system 200 presented herein may be used in any desired environment. Thus FIGS. 2A-2B (and the other FIGS.) should be deemed to include any and all possible permutations.

Looking to FIGS. 2A-2B, the ultrasound probe 203 includes a housing 202, and an array of transducers 204 positioned at a contact head of the 201 ultrasound probe 203. As described above, the transducers 204 transduce reflected sound waves into electrical signals. Thus, the transducers 204 preferably include piezoelectric devices, e.g., of a type known in the art.

Moreover, the housing 202 is preferably configured for grasping by a human hand. In other words, the housing 202 preferably implements dimensions that allow for a human hand to pick-up and use the ultrasound probe 203 via the housing 202. An illustrative dimension of the housing 202 may include an average outer diameter D along a longitudinal axis thereof that is from about 1.5 inches to about 3 inches. The housing 202 also preferably has a cylindrical shape, but in other embodiments, the housing 202 may have a different shape, e.g., a rectangular shape. A length L of the housing 202 may be from about 4 to about 6 inches, but may be higher or lower depending on the desired embodiment. Furthermore, a width W of the contact head 201 may be from about 1.5 inches to about 4 inches, but may be higher or lower depending on the configuration of the contact head 201, as will be described in further detail below.

However, the housing 202 is also preferably configured to receive a circuit board 206. Thus, a circuit board 206 may be positioned in the housing 202. The circuit board may be a single board, or a plurality of boards operating together, e.g., for providing some feature of the overall design.

The characteristics of the circuit board 206 may be selected depending on the dimensions of the circuit board 206 and/or a cavity formed by the housing for receiving the circuit board 206. For example, if the circuit board 206 has dimensions smaller than that of a cavity formed by the housing 202, the circuit board 206 may be made of a rigid material, e.g., any substrate material that would be apparent to one skilled in the art upon reading the present description. However, if the dimensions of the circuit board 206 are larger than that of a cavity in the housing 202, the circuit board 206 may be constructed using a flexible material of a type known in the art. In such embodiments, the flexible circuit board may be folded, rolled, curled, etc. to fit in the cavity of the housing 202, which may result in space savings of up to 75% over rigid circuit boards.

Moreover, by implementing a flexible circuit board 206, package designers are given the freedom to relocate various components and/or subassemblies to locations that may further optimize circuit performance and/or system operation. In other words, designers are no longer restricted by the space demands of rigid PC boards. Furthermore, simplifying circuit geometry and placing surface mount devices directly on the circuit board 206 may also improve the circuit design. Intricate patterns that may be difficult to achieve with rigid board connector pins can be designed into the flexible circuit board configurations. Thus, greater circuit complexity is achieved in a much smaller space.

Referring still to FIGS. 2A-2B, the circuit board 206 may have a plurality of leads 208, each of the transducers 204 being coupled to at least an associated one of the leads 208. The leads 208 may include any desired material, one or more of which may provide an electrical connection between the transducers 204 and at least a portion of the circuit board 206.

In one approach, the leads 208 may be printed onto a flexible circuit board using conventional circuit printing technology. In another approach, the leads may be formed by etching through a conductive overlayer of a flexible circuit board to define the leads.

The circuit board 206 further includes processing circuitry 210 coupled thereto, e.g., using etching, or any other method known in the art. The processing circuitry 210 is preferably used for processing electrical signals received from the transducers 204 via the leads 208. Moreover, the processing circuitry 210 may be able to additionally and/or alternatively process derivatives of the electrical signals received from the transducers 204 via the leads 208. According to a preferred approach of the present description, “processing” is intended to mean that the processing circuitry 210 is able to process electrical signals received from the transducers 204 and convert such electrical signals into sonogram data.

The inclusion of the leads 208 and processing circuitry 210 on the circuit bard 206 itself eliminates the need for cabling to the back-end computing device 214.

Thus, processing done to the electrical signals received from the transducers 204 is performed on the wireless ultrasound probe 203. As a result, sonogram data is produced on the wireless ultrasound probe 203 before being sent off the probe 203, e.g., to a back-end computing device 214.

The ultrasound probe 203 includes an output device 212 for outputting the sonogram data produced by the processing circuitry 210 to a back-end computing device 214. As mentioned above, and illustrated in FIG. 2A, the ultrasound probe 203 need not be physically connected to a back-end computing device 214, e.g., at least during use. Rather, the ultrasound probe 203 may be wirelessly connected a back-end computing device 214. Thus, according to different approaches, the output device 212 may include a wireless transmitter that uses wifi, BlueTooth, etc. The wireless connection between the ultrasound probe 203 and back-end computing device 214 may be direct, via a wireless network, etc.

By implementing an ultrasound probe 203 that is capable of wirelessly operating and relaying information to a back-end computing device 214, the ultrasound probe 203 is not constrained to being used within a range defined by a physical cable. Moreover, production and upkeep costs are dramatically reduced as the leads 208 connecting the transducers 204 to the circuit board 206 are located in the housing of the ultrasound probe 203 itself. Thereby the leads 208 are shorter than conventional micro-coaxial cables by orders of magnitude.

According to an exemplary in use embodiment, the array of transducers 204 may include 64 piezoelectric transducer elements. Moreover, by implementing the approaches described herein, the 64 piezoelectric transducer elements may produce a resolution visually identical to a conventional system having 128 piezoelectric transducer elements. In one embodiment, 512 by 512 pixel image data is producible from the sonogram data developed by the processing circuitry 210 in the ultrasound probe 203.

According to one approach, the 512 by 512 pixel image may be displayed in greyscale on the back-end computing device 214. Accordingly, each pixel is represented by 8 bits, e.g., corresponding to an 8 bit greyscale. Moreover, the image data may be updated at 30 frames per second, thereby resulting in a data rate of 512×512×8×30=63 Mbps from the ultrasound probe 203.

Alternatively, according to another approach, the 512 by 512 pixel image may be displayed in color. Accordingly, each pixel is represented by 24 bits, e.g., corresponding to the RGB color scheme. As mentioned above, the image may be updated at 30 frames per second, thereby resulting in a data rate of 512×512×24×30=118 Mbps.

The aforementioned data rates represent illustrative raw bandwidths of the sonogram data developed by the processing circuitry 210 in the ultrasound probe 203. Such raw data may be sent to the back-end system 214. However, such large amounts of data may require significant amounts of power and/or computing, not to mention transmission resources. Thus, the data rates may be compressed to less than about 1/63^{rd} of the original bandwidth in the probe 203, or in the back-end system 214 for retransmission to a remote system. For example, grayscale imaging may be compressed and transmitted at about 800 kbps, while RGB color imaging may be compressed and transmitted at about 3 Mbps.

Referring still to FIGS. 2A-2B, although the ultrasound probe 203 is preferably wirelessly connected a back-end computing device 214, the output device 212 may include a universal serial bus (USB) interface. Depending on the desired embodiment, the USB interface may be compatible with the USB 2.0 standard, the USB 3.0 standard, the USB 3.1 standard, etc. Depending on the desired embodiment, the USB interface may provide for a supplemental method of connecting the ultrasound probe 203 to a back-end computing device 214.

According to an in-use embodiment, which is in no way intended to limit the invention, the ultrasound probe 203 may deactivate its output device 212, e.g., to conserve energy in a low power state, when out of range from a back-end computing device, etc. Moreover, when the output device 212 is deactivated, information pertaining to signals receive from the transducers 204 and/or sonogram data derived therefrom may be stored on the probe 203 itself until the output device 212 is reactivated. However, while the output device 212 is deactivated, the USB interface of the output device may be coupled to a USB interface of the back-end computing device 214 using a temporary physical connection, e.g., a cable. Thus, information pertaining to signals receive from the transducers 204 and/or sonogram data derived therefrom may be transferred from the ultrasound probe 203 to the back-end computing device 214, e.g., for further processing and/or analysis.

In additional embodiments, the output device 212 may include an ethernet interface, or any other interface that may facilitate a detachable, electrical connection between the output device 212 of the ultrasound probe 203, and a back-end computing device 214. Moreover, according to yet another embodiment, the output device 212 may include both a USB interface and an ethernet interface.

It follows that, because the ultrasound probe 203 is wireless, the ultrasound probe 203 may further include a battery 228 for powering the processing circuitry 210 and/or any other components in the ultrasound probe 203. Depending on the desired embodiment, the battery 228 may include any type of battery apparent to one skilled in the art armed with the present teachings. Thus, the ultrasound probe 203 may include one or more replaceable batteries or receptacle therefor, one or more rechargeable batteries, etc. Ultrasound probes having one or more rechargeable batteries may use any type of recharging scheme known in the art. In one approach, a USB interface may be used to recharge said one or more batteries when coupled to a power source via a cable electrically coupled to the USB interface.

In some embodiments, the ultrasound probe 203 may further include a heat sensor 216 coupled to the housing 202 using any desired means of coupling the sensor 216 thereto. The heat sensor 216 may be of any type known in the art, and preferably monitors the temperature of an outer surface of the housing 202 and/or contact head 201 of the ultrasound probe 203. As described above, the ultrasound probe 203 may be used to generate ultrasonic images of human and/or animal patients. Therefore, it is preferred that the ultrasound probe 203 does not reach temperatures that may irritate, burn, etc. the patient being examined or the person holding the ultrasound probe 203. It follows that the heat sensor may be coupled to a temperature tracking device, e.g., on the circuit board 206, that may monitor the temperature of the housing 202 and/or contact head 201. The temperature tracking device may detect when the temperature of the housing 202 and/or contact head 201 pass a temperature threshold, and as a result, warn a user, automatically turn off the ultrasound probe 203, automatically turn off the output device 212, etc. Moreover, the temperature threshold may be predetermined by a user, stored in a lookup table, etc. An illustrative temperature threshold may be about 40 degrees Celsius, but may be higher or lower, depending on the desired embodiment.

Although the embodiment of FIG. 2A illustrates an ultrasound probe 203 having a contact head 201 according to a particular embodiment, in other embodiments, the ultrasound probe 203 may have a different contact head 201. In one approach, the contact head 201 of the ultrasound probe 203 may be detachable, and preferably interchangeable with other contact heads. Depending on the desired embodiment, a contact head may be selected from a group consisting of a linear array configuration, a micro-convex configuration and a phased array configuration. However, further embodiments may include additional configurations.

Moreover, as described above, a width W of the contact head 201 may be from about 1.5 inches to about 4 inches, but could be higher or lower. Furthermore, a length L₂ of the contact head 201 may be from about 1.5 inches to about 3 inches, but could be higher or lower depending on the desired embodiment.

Looking to FIG. 2C, an exemplary ultrasound probe kit 260 is illustrated. It should be noted that the embodiment of FIG. 2C is intended to include all the features of FIGS. 2A-2B and additionally incorporate different interchangeable contact heads, as briefly described above. It follows that various components of FIG. 2C have common numbering with those of FIGS. 2A-2B.

The kit 260 includes different contact heads 201 that are selectively detachable from a housing 202. Contact heads 201 in the kit 260 according to the present embodiment include a contact head having a linear array configuration 262, a contact head having a micro-convex configuration 264 and a contact head having a phased array configuration 266. The housing 202 and contact head 201 may be selectively detachable by using a retractable pin, friction, etc. Moreover, kits including different combination of contact head configurations may be implemented in other embodiments. The array of transducers may remain in the housing, or may be in the contact head.

Depending on which contact head configuration is attached to the ultrasound probe 203, a user may be able to adjust the functionality of the ultrasound probe 203 accordingly. For example, if a contact head 201 having a linear array configuration is detached from the ultrasound probe 203 and a contact head 201 having a micro-convex configuration is then attached to the ultrasound probe 203, the ultrasound probe 203 preferably includes a control (e.g., see 222 of FIG. 2B) to adjust the functionality of the ultrasound probe 203 from a setting corresponding to a contact head 201 having a linear array configuration to a setting corresponding to the now attached contact head 201 having a micro-convex configuration.

Referring specifically now to FIG. 2B, an exemplary circuit diagram 250 of the circuit board 206 is depicted in accordance with one embodiment. As described above, the circuit board 206 may be one or more flexible circuit boards. Moreover, the circuit board 206 includes a power switch 220, USB port 230, an array of transducers 204 and beamformer 104. In some approaches, USB port 230 may function as an output device, e.g., supplementing output device 212 of FIG. 2A as previously mentioned. Furthermore, circuit diagram 250 also includes processing component 102 which may include an embedded central processing unit (CPU), and memory 234 which may be any conventional type of memory, e.g., random access memory (RAM), flash memory, etc.

AFE 236 is also positioned on the flexible circuit board 206. According to various embodiments, AFE 236 may include any number of digital to analog converters (e.g., see 106 of FIG. 1B), voltage generators, multiplexers, analog to digital converters, etc.

The circuit diagram 250 further includes a battery 228, which may include any of the battery types described and/or suggested in any of the approaches above. Furthermore, a direct current (DC) to DC converter 232 and surge protector fuse 240 are coupled to the battery 228. DC to DC converter 232 and surge protector fuse 240 may include any conventional components, e.g., depending on the desired embodiment. However, it is preferred that the surge protector fuse 240 prevents any damage from being done to the DC to DC converter 232, the battery 228 and/or any components in the AFE 236, e.g., resulting from a voltage surge. As described above, the transducers may be actuated with sequences of high voltage pulses that may originate from voltage generators positioned in the AFE 236. It follows that the surge protector fuse 240 may be configured to police such voltage pulses and/or any surges, e.g., to prevent any damage to circuitry.

Referring still to FIG. 2B, DC to DC converter 232 is also coupled to transmit beamformer 238. Transmit beamformer 238 preferably focus the array of transducers 204 for forming a signal to be transmitted. According to an exemplary embodiment, the transmit beamformer 238 may change the phase and relative amplitude of the signal to control the directionality of the signal when transmitting. In further approaches, the transmit beamformer 238 may additionally or alternatively include any conventional functionality as would be appreciated by one skilled in the art upon reading the present description. Moreover, transmit beamformer 238 may include any of the features described above for beamformer control unit 104 of FIG. 1B.

Circuit diagram 250 of the exemplary wireless ultrasound probe in FIG. 2B further includes a light source 224, e.g., for illuminating an environment near the housing 202. According to various approaches, the light source 224 may include one or more LEDs, halogen bulbs, lasers, etc. Moreover, light source 224 may be operated (turned on and off) using one of the controls 222, 226, the power switch 220, and/or a separate switch/control. In some approaches, the light source may be detachable.

As described above, an ultrasound probe preferably includes a control to adjust the functionality of the ultrasound probe depending on the contact head configuration coupled thereto. Thus, looking again to the circuit diagram 250 a control 222 is included for selecting an operational mode selected from a group consisting of a linear array mode, a micro-convex mode and a phased array mode. However, further embodiments may include additional operational modes, e.g., corresponding to different contact head configurations. Referring still to FIG. 2B, the control 222 may include any type of user interface for selecting an operational mode of a corresponding ultrasound probe.

Similarly, the circuit diagram 250 includes a second control 226 on the housing for adjusting at least one of a frequency and depth of the sound waves generated by the array of transducers 204. As previously mentioned, the number of sound waves emitted determines the resulting field of view, while the frequency of the emitted sound waves determines the resolution of the resulting image generated from the reflected sound waves and the power of the emitted sound waves determines how deep the sound waves penetrate. According to the present description, the desired depth, resolution and/or field of view may vary depending on the in-use application. Thus, the second control 226 may allow for a user to adjust the frequency and/or the depth of the sound waves generated. It should also be noted that although control 222 and second control 226 are shown as different components in the present embodiment, in other approaches, the control 222 and second control 226 may be incorporated into a single control.

A number of supplemental embodiments are provided below which are intended to be presented by way of example only, and are in no way intended to limit the invention. It follows that any of the exemplary supplemental embodiments presented below may be implemented in conjunction with features from any other embodiment listed herein, such as those described with reference to the other FIGS. Of course, however, such supplemental embodiments and others presented herein may be used in various applications and/or in permutations which may or may not be specifically described in the illustrative embodiments listed herein. Further, the supplemental embodiments presented below may be used in any desired environment.

Looking to FIG. 3, a single element piezoelectric transducer 300 is illustrated according to one embodiment. As an option, the present transducer 300 may be implemented in conjunction with features from any other embodiment listed herein, such as those described with reference to the other FIGS. Of course, however, such transducer 300 and others presented herein may be used in various applications and/or in permutations which may or may not be specifically described in the illustrative embodiments listed herein. Further, the transducer 300 presented herein may be used in any desired environment. Thus FIG. 3 (and the other FIGS.) should be deemed to include any and all possible permutations.

Referring now to FIG. 3, transducer 300 includes an outer casing 302, backing material 304, electrodes 306, piezoelectric crystal 308, acoustic lens 310, acoustic insulator 312 and cable 314.

Ultrasound examinations are of little value in examining organs that contain air because ultrasound waves will not pass through air and therefore they cannot be used to examine normal lungs. Bone also stops ultrasound waves, so the brain and spinal cord are unable to be seen with an ultrasound study, and obviously, bones cannot be examined. Depending on the images produced, ultrasound can take various forms. In veterinary work brightness-mode (B-mode) ultrasound, more commonly called 2-dimensional ultrasound, is the most common form. This gives a two dimensional picture of the organ scanned. This type of ultrasound is preferred when examining abdominal structures, perform pregnancy diagnosis, evaluate cardiac function and examine the eyes for certain eye diseases.

Motion-mode (M-mode) is a type of B-mode in which a tracing of the motion of the structure being scanned is displayed. A combination of M-mode and 2-dimensional ultrasound may desirably be used for examining the heart walls, chambers and valves to evaluate cardiac function.

Cardiac ultra-sonography is usually referred to as echocardiography. Doppler ultrasound is a specialized form of cardiac ultrasound in which the direction and speed of blood flow in the heart and blood vessels can be measured. Color-flow Doppler technology makes it even easier to observe the flow of blood through the heart and important blood vessels.

FIG. 4 illustrates a representational diagram of an AFE 400 according to one embodiment. As an option, the present AFE 400 may be implemented in conjunction with features from any other embodiment listed herein, such as those described with reference to the other FIGS. Of course, however, such AFE 400 and others presented herein may be used in various applications and/or in permutations which may or may not be specifically described in the illustrative embodiments listed herein. Further, the AFE 400 presented herein may be used in any desired environment. Thus FIG. 4 (and the other FIGS.) should be deemed to include any and all possible permutations.

As illustrated, AFE 400 includes amplifier 402, multiplexer 404, transmit and receive (T/R) switch 406 and transmit beamformer 408. AFE also includes LNAs 414, time gain controls 416 and analog beamformer 418. Moreover, multiplexer 404 is connected to transducers 410 via cable 412.

The transmit beamformer 408 may be responsible for the orderly pulse-excitation of transducers 410, which results in emission of acoustic waves into a region of interest. Moreover, the T/R switch 406 may be used to switch the front end into a receive mode. Receive mode preferably corresponds to transducers 410 transforming the reflections or echoes of the emitted acoustic waves, into corresponding electrical signals. The AFE 400 properly amplifies these signals and converts them into digital data streams, e.g., for further processing. By applying dynamic delays into these data streams, the receive beam former combines them to form a scan line, a representation of the region of interest along a given line of sight.

The aforementioned functionality of the AFE 400 may be repeated either sequentially or simultaneously to form multiple scan lines to cover a given region of interest. In such embodiments, a front-end controller (not shown) may be responsible for controlling the timing and sequencing of transmit and receive beams. Sampling rates used for analog-to-digital (A/D) conversion in the front-end controller may vary from about 16 MHz to about 50 MHz, depending on system requirements of the desired embodiment.

Furthermore, depending on the desired embodiment, the AFE 400 may be implemented in an ultrasound system, e.g., as illustrated in FIGS. 1A-1B.

Looking now to FIG. 5, a representational diagram of the digital front end 500 of an ultrasound system is illustrated according to one embodiment. As an option, the present digital front end 500 may be implemented in conjunction with features from any other embodiment listed herein, such as those described with reference to the other FIGS. Of course, however, such digital front end 500 and others presented herein may be used in various applications and/or in permutations which may or may not be specifically described in the illustrative embodiments listed herein. Further, the digital front end 500 presented herein may be used in any desired environment. Thus FIG. 5 (and the other FIGS.) should be deemed to include any and all possible permutations.

In some embodiments, digital front end 500 may be used to generate a digital beam as would be appreciated by one skilled in the art upon reading the present description. Looking specifically to FIG. 5, the digital front end 500 includes.

Ultrasound beamformers as used herein, may include two parts. A first part may include a transmit beamformer (Tx beamformer). In different approaches, the Tx beamformer may be responsible for initiating scan lines and/or generating the timed pulse string to the transducer elements to set the desired focal point of the subject. Moreover, a second part of the ultrasound beamformers may include a receive beamformer (or Rx beamformer). The Rx beamformer may be responsible for receiving the echo waveform data from the analog front end, and collating the data into representative scan lines through filtering, windowing (apodization), summing, and/or demodulation. Tx and Rx beamformers may further have blocks that are time synchronized and/or continuously pass timing, position, control data, etc. to each other.

The Tx beamformer may be responsible for steering and generating a timed, digital pulse string that may then be externally converted into high-voltage pulses compatible with the transducers. The delay may be calculated in real-time, based on the required instantaneous location of the focused ultrasound beam for the given scan line. This operation corresponds to a fairly small block, e.g., requiring less than about 10% the logic resource of the Rx beamformer. Depending on the approach, it may include a timing generator and/or pulse shaping, and typically has a parallel interface to external DACs.

The Rx beamformer parses the raw transducer Rx data to extract and assemble ultrasound scan lines. In preferred approaches, it is a DSP intensive block that consumes a large amount of logic resources. Each step up to summation may be performed per channel, while the remaining steps may be performed per scan line. Rx beamforming can be performed in the frequency domain, time domain, etc., or using other proprietary methods which may include, but are not limited to, any of the following:
Data Capture—Deserializes the incoming data, synchronizes the clocks, and buffers the data for processing.
Sample—Oversamples the incoming data to enable better accuracy in the subsequent delay process.
Interpolation filter—Helps to improve image accuracy by further upscaling and adjusting for delay inaccuracies.
Delay/Focus—Data is delayed on each channel to adjust for the position of the focal point relative to each transducer receive element. The timing here is synchronized with the Tx beamformer and can be altered by the system user in real time to steer the beam and focal point.
Windowing/Apodization—Removes spatial image echoes (side lobes) that naturally occur in a beam response.
Summation—Sums all the channels together to create final scan line representation.
Demodulation—Demodulation extracts the final scan line from the echo carrier frequency range. This process often includes envelope detection, down conversion, decimation filters, and matched filters. Hilbert transform is typically used for envelope detection.
Logarithmic Compression—Reduces the dynamic range of the data to acceptable levels for image processing and display.

It should be noted that the foregoing list of proprietary methods are in no way intended to limit the invention, but rather are presented by way of example.

FIG. 6 depicts a representational diagram 600 of a digital back end processing engine according to one embodiment. As an option, the present diagram 600 may be implemented in conjunction with features from any other embodiment listed herein, such as those described with reference to the other FIGS. Of course, however, such diagram 600 and others presented herein may be used in various applications and/or in permutations which may or may not be specifically described in the illustrative embodiments listed herein. Further, the diagram 600 presented herein may be used in any desired environment. Thus FIG. 6 (and the other FIGS.) should be deemed to include any and all possible permutations.

Referring now to FIG. 6, the representational diagram 600 of a digital back end processing engine includes spectral Doppler processing (D-mode) 602, image and motion processing (B-mode) 604, color Doppler processing (F-mode) 606, display 608 and audio output 610.

Back end processing engines typically include B-mode, M-mode, Doppler, and color flow processing functions. B-mode operation produces a gray scale image that may be used for examining tissue structures and organs. Color-flow operation produces a color-coded display of spatial distribution of mean velocity of blood flow super-imposed on gray scale image. Moreover, Doppler processing produces scrolling display of blood flow velocity distribution at a user specified location. Common to all three is the initial stage where beam formed data gets down converted to baseband.

B-mode operations includes envelope detection and logarithmic compression. For color flow to occur, high pass filtering of ensembles of scan lines is desired, e.g., to remove contributions from vessel wall or tissue motion. Moreover, B-mode and color-flow estimates may be subjected to temporal and spatial processing, e.g., to reduce noise and enhance features of interest. In some embodiments, scan conversion includes B-mode and color-flow estimates which may be converted to display raster data, pixels with 1:1 aspect ratio. When color-flow is on, B-mode and color-flow pixels are desirably blended to produce a single image. This blending is typically based on application dependent thresholds. Furthermore, some systems may be capable of displaying three modes simultaneously.

However, Doppler processing may use a much simpler wall filter and estimation of velocity distribution using short-time Fourier transform techniques. Doppler processing also produces a stereo audio signal representing the Doppler spectrum.

Similar to most embedded systems, ultrasound imaging systems preferably include a system controller to carry out various functions which may include, but are not limited to:
Configuring and controlling the signal path.
Handling user input events and taking appropriate actions.
Monitoring acoustic pressure and intensity levels and ensuring safety of patients.
Carrying out smart power management to maximize scanning time in a single charge.
Storing and recalling image clips.
Running applications to allow you to make clinically relevant measurements on acquired image sequences.

As described above, ultrasound diagnostics are desirably as they are non-invasive and cause no damage to the organic patient (e.g., human and/or animal bodies). The common ultrasound diagnostic examinations include the following test areas:
Abdomen: evaluation of soft tissues, blood vessels and/or organs of the abdominal cavities (e.g., liver, spleen, urinary tract, pancreas, etc.).
Obstetrics/Gynecology: evaluation of the female reproductive system and/or a fetus.
Echocardiography: (adult echo, pediatric echo, fetal echo) evaluation of the anatomy and/or hemodynamics (blood flow) of the heart, its valves and related blood vessels.
Vascular Technology: evaluation and analysis of the hemodynamics (blood flow) or cerebral peripheral and/or abdominal blood vessels.
Neurosonology: evaluation of the brain and/or spinal cord.
Breast: frequently used to evaluate breast abnormalities that are found through screening or diagnostic mammography, especially to differentiate breast cysts (benign) from potentially cancerous growths.
Ophthalmology: evaluation of the eye, e.g., including orbital structures and/or muscles.

It follows that imaging modality achieved using any of the embodiments described and/or suggested herein is desirably able to achieve an accurate representation of the internal organs of a human or animal body. Moreover, embodiments described and/or suggested herein are also desirably able to determine movement within the body (e.g., blood flow), using Doppler signal processing. From this information a doctor may then make conclusions about the correct functioning of a heart valve or blood vessel.

According to various embodiments, a probe according to any of the approaches described herein may include one or more of the following features:
Cine Loops selectable from 32 frames to 512 frames
256 shades of gray
Supported Depths: 6 CM, 10 cm, 15 cm, 20 cm
Image resolution of 1 mm
Every probe is water resistance
Automatic patient data base for images (stills and cine loops) and reports
Power on/off button
Preset function
Image Optimization Control
Depth Control
Gain Control
Freeze Control
Time Gain Compensation
Ultrasound Mode Selectors
Patient: enters the patient information as a patient chart; some systems can be programmed to auto select patient ID
Speckle Reduction
Sound Speed Correction
90 degree sector size and possibly 270 degree sector size
256 scan vectors per scan, 512 samples per vector

It should be noted that the foregoing list of features are presented by way of example only and are in no way intended to limit the invention.

Looking now to FIG. 7, an ultrasonic probe 700 is illustrated according to one embodiment. As an option, the present probe 700 may be implemented in conjunction with features from any other embodiment listed herein, such as those described with reference to the other FIGS. Of course, however, such probe 700 and others presented herein may be used in various applications and/or in permutations which may or may not be specifically described in the illustrative embodiments listed herein. Further, the probe 700 presented herein may be used in any desired environment. Thus FIG. 7 (and the other FIGS.) should be deemed to include any and all possible permutations.

The probe 700 includes an array of transducers 702 and backing material 704 which implement a screwable transducer head 706 having 3 male screw threads in the present embodiment. The array of transducers 702 may be a linear, micro-convex phased piezoelectric array. Moreover, the array of transducers 702 may correspond to 64 channels.

Flexible PCBs 708 are also positioned in the probe 700, in addition to multi-function buttons 710, power strip 712 and battery compartment 714. Probe 700 further includes screw threads 716 in the battery housing, battery 718, positive electrode 720 of the battery 718, power button 722, function LEDs 724, terminating strips 726 and a flashlight 728.

Illustrative dimensions of probe 700 and its different components are also presented in FIG. 7, but are in no way intended to limit the invention. For example, a length of the transducer head 706 may have a length from about 1 inches to about 3 inches, while the main body of the probe 700 may have a length from about 4 inches to about 6 inches, and the battery compartment 714 may have a length of about 2 inches to about 4 inches, but may be higher or lower depending on the desired embodiment. Moreover, a width of the probe 700 may be from about 1.5 inches to about 2.5 inches, but may be higher or lower.

Moreover, the foregoing features preferably ensure that the screwable heads (e.g., see 706 of FIG. 7) do not create any unnecessary reflections and/or degradation in signal strength. Furthermore, screw threads as used herein are preferably designed to be fully water tight and may be screwed on and off with little effort from a user. Moreover, transducer array 702 and/or transducer head 706 may further be protected by backing layer made of a backing material 704. The backing material 704 supporting the crystal may have a substantial influence on the damping characteristics of a transducer array 702. Furthermore, the backing material 704 used preferably has similar matching impedance as to that of the active element, e.g., in order to produce a highly effective damping. As a result, wider bandwidth may be achieved, further resulting in higher sensitivity. According to one approach, the backing material 704 may be made of rubber and encapsulated in epoxy resin.

As mentioned above, the screwable head 706 has threads that screw into the probe casing 730. The threads are preferably constructed such that they may be easily screwed off and on and the thread is strong to have mean time before failure (MTBF) for at least 3 years. According to some embodiments, screwable heads may be used instead of a clip on or punch down style coupling mechanism is for robustness and resiliency. Even if the probe is dropped, the transducer head 706 will not become detached from the probe casing 730.

The probe 700 includes four flex PCBs 708 as shown. Use of flexible PCBs in the probe not only provides higher functionality within limited space, but it also reduces the weight of the overall probe 700 as the flexible PCBs 708 are light weight compared to rigid PCBs. The PCB nearest to the transducer head 706 has electronics that make up an analog front end of the probe 700. The PCB nearest the transducer head 706 is also connected to the beam forming digital front end PCB which in turn is connected to the digital backend PCB. The fourth flex PCB in the present embodiment may include an IEEE 802.11n/ac WiFi module that wirelessly connects the probe 700 to the Windows based host system that could be a desk top, lap top or a tablet. Moreover, in further approaches, the probe 700 may also be able to communicate with IOS and Android based computing systems that support IEEE 802.11n/ac interfaces. Additionally, the probe 700 may include space available to include another interface, e.g., as a backup, which may be a wired connection that could be USB 3.0, Ethernet, etc.

Embodiments implementing IEEE 802.11n/ac functionality may be able to meet the data rates desired to achieve 30 frames per second and/or for 60 frames per second at distances of up to 5 m. According to other exemplary embodiments, USB 3.0 may be capable of generating enough bandwidth to support both gray scale and color transmission of data. Further still, some embodiments may use a 1 Gbps Ethernet interface.

Referring still to FIG. 7, a battery compartment 714 is located at a bottom portion of the probe 700. The battery compartment 714 houses a battery 718 which, according to an exemplary embodiment, may include a 3000 mAH rechargeable battery, e.g., that may power the probe 700 for up to about 1 hour. The battery compartment 714 is preferably built in such a way that the battery 718 is sturdily coupled to the battery compartment 714. Even with rough handling and dropping of the probe 700, the battery 718 will not disconnect from its electrodes 720. As mentioned above, the battery compartment 714 screws on to the fuselage, e.g., similar to the functionality of a flashlight. The battery compartment 714 is also preferably built from industrial strength plastic, e.g., having a MTBF of about 3 years.

The probe 700 also has 4 buttons (i.e., multi-function buttons 710 and power button 722) and 2 LEDs 724. The multi-function buttons 710 and the LEDs 724 are preferably multi-functional. According to an exemplary embodiment, one of the LEDs 724 may denote a power state (e.g., on or off) while the other LED 724 may be reserved to denote the status of the multi-functional buttons 710. The power button 722 is preferably separated from the other multi-function buttons 710, e.g., to ensure that the power button 722 is not pressed inadvertently when performing an examination. Once the power button 722 is switched to an “on” position, the LED 724 denoting the power condition of the probe 700 may emit a solid green light, e.g., to denote that the probe 700 is on (functional). Moreover, if there is any problem with the power supply (e.g., low battery charge level, faulty electrical connections, etc.), the LED 724 denoting the power condition of the probe 700 may flicker while emitting an amber color. Furthermore, power failure may be denoted by the power condition LED 724 turning solid red.

The multi-function buttons 710 include three distinct function buttons on the probe 700. In one approach, each of the multi-function buttons 710 may be a different color. According to a preferred approach, the multi-function buttons 710 are positioned towards a bottom portion of the probe 700, e.g., towards the battery compartment 714, as shown in the FIG. 7.

According to an exemplary in-use embodiment, which is in no way intended to limit the invention, once the probe 700 is powered on, e.g., using the power button 722, the lap top or the host system connected thereto may prompt the user (e.g., sonograph technician) to log on. A drop down menu may appear on a screen of a graphical user interface of the host system that may prompt the user to prepare for the examination.

Of the multi-function buttons 710, the button closest to the transducer head 706 may correspond to measurement control, while the multi-function button closest to the battery compartment 714 may be reserved for patient type selection, e.g., to select whether human or animal is being examined. Moreover the middle button may correspond to the examination type (B-Mode, M-Mode, etc.).

Pressing (e.g., activating) the button closest to the transducer head 706 twice may prompt the drop down menu to appear on screen of the graphical user interface of the host system as described above. Moreover, in one approach, the drop down menu may list the type of examination that the user (e.g., sonograph technician) wishes to conduct. According to different embodiments, the selections may include any of the following:
Obstetrics, Early Obstetrics, Gynecology, Abdomen. Renal, Urology, Fetal Echo, Emergency Medicine, Peripheral Vascular, Venous, etc., having operational frequencies from about 2 MHz to about 5 MHz, using linear or micro-convex type contact heads.
Cerebrovascular, Peripheral Vascular, Thyroid, Testicle, Breast, Musculoskeletal, Venous, Orthopedic, Emergency Medicine, etc., having operational frequencies from about 5 to about 10 MHz, using linear or micro-convex type contact heads.
Cardiac, Abdomen, Renal, Gynecology, Obstetrics, Transcranial, Emergency Medicine.
Pediatric Abdomen. Renal, Pediatric Echo, Neonatal, etc., having operational frequencies from about 2 MHz to about 8 MHz, using a phased array contact head.

Once the user (e.g., sonograph technician) selects the type of exam to be conducted using a feature of a graphical user interface, e.g., the keyboard of a host computer, the user may press the middle button twice and a drop down menu may appear on the screen of the graphical user interface of the host system for selecting the mode.

On confirming the type of exam by selecting on the display, the probe (with its in built expert systems) may automatically adjusts one or more of the settings to optimize examination settings. The probe may store information corresponding to optimized examination settings in the flash memory.

The second LED may emit a solid green light to indicate that all parameters are set correctly and the user is ready to conduct the exam. Once the green LED is lit up the pressed buttons may be released back to the original mode. Moreover, during the exam, if the user wants to freeze an image produced using the probe, the user may press one of the multi-function buttons of the probe one time, and that may desirably freeze the image on the screen of the graphical user interface on the host system.

The user (e.g., sonograph technician) may further be able to mark the frozen image on the screen of the graphical user interface on the host system, e.g., to denote a suspicious spot. Furthermore, the graphical user interface may allow for the image displayed on the screen to be zoomed into the suspicious spot for further diagnosis. Once the user has thoroughly examined the suspicious spot, e.g., to determine what the symptom may be, the user may release the middle button which may thereby unfreeze the image displayed on the screen of the graphical user interface.

Flashlight 728 may be added in some embodiments. Although it may be permanently attached to the casing 730 of the probe 700, in preferred embodiments, flashlight 728 has a clampable or ring based 4 lumen flashlight to view the examination more clearly, e.g., poorly lit areas (especially outdoors).

In some approaches, the whole probe casing may be water proof, e.g., up to 30 meters. Moreover, depending on the desired embodiment, the probe casing may have any one or more of the following physical characteristics:
Direct WiFi interface between probe and host
Up to 9 inches length and 2 inches in diameter
1 hour battery operation
3 inches long and 2 inches in diameter battery compartment
2 inches long and 2 inches in diameter transducer head
Circular buttons embedded in the fuselage (0.3″ diameter)
2 LEDs embedded on top of fuselage close to the power button (0.1″ diameter)
Operating temperature probe and battery 0 to 50° C.
Storage temperature probe −16° C. to 60° C.
Storage temperature battery −20° C. to 60° C.

In other embodiments, an ultrasound probe may include a control panel, e.g., to assist a user in controlling the probe. In such embodiments, the control panel may include any number of buttons, switches, cursors, sliding scales, etc., that are known in the art. According to various approaches, the control panel may include any of the following buttons:
Power on/off button: turns the ultrasound system on or off
Preset: allows one to select the appropriate preselect for scanning: example ob-gyn, nerves or small parts
Image Optimization Control: changes the frequency of the probe for optimum penetration and resolution of the scan. Higher frequency will give better resolution and lower frequency will give better penetration
Depth Control: changes the field of view for the area being examined
Gain Control: adjusts the acoustic power of the transmitted signal
Freeze Control: freezes the image for acute evaluation. Once complete, pressing again unfreezes the image
Time Gain Compensation: these are normally arranged as sliders. Each of the sliders adjust the amplification of the echo in 2D mode at a specific depth
Ultrasound Mode Selectors: B-Mode, M-Mode. Pulsed Doppler, Color Doppler
Imaging/Measurement Key: cursor, clear, body mark, measure, M/D cursor, scan area, set/pause, depth/zoom/ellipse
Patient: enters the patient information as a patient chart: some systems can be programmed to auto select patient ID
Speckle Reduction: reduces unwanted speckle noise from the image
Sound Speed Correction: the resolution in the lateral dimension deteriorates due to a difference in sound speed. By correcting this and carrying out optimization, the resolution in the lateral dimension is improved

It should be noted that the foregoing list of potential buttons of a control panel are in no way intended to limit the invention, but rather are presented by way of example. Moreover, any of the foregoing buttons may be included on a control panel coupled to any of the ultrasonic probes and/or systems described herein.

Transducers used for the transducer arrays may be custom built to meet desired specifications, e.g., to achieve optimum performance of a corresponding transducer head. The piezoelectric crystal is cut with precision to generate an array of 64 elements that takes into consideration material, mechanical and electrical construction, and the external mechanical and electrical load conditions. Mechanical construction may include parameters such as the radiation surface area, mechanical damping, housing, connector type and other variables of physical construction. In an illustrative approach, the piezoelectric crystals may be cut to a thickness that is ½ the desired radiated wavelength.

To increase energy output of the transducers, optimal impedance matching may preferably be achieved by sizing the matching layer so that its thickness is about ¼ of the desired wavelength. This keeps waves that were reflected within the matching layer in phase when they exit. Moreover, the backing material supporting the crystal may have a substantial influence on the damping characteristics of one or more of the transducers. The backing material used preferably has similar matching impedance as to that of the active element in order to produce the most effective damping. As a result, this may then produce a wider bandwidth resulting in higher sensitivity.

The 64-elements of the transducer arrays are arranged on a plane (linear array) or a curved surface (curved array). Moreover, the electrical wires from each element are preferably transmitted on the flex PCB terminal block as described above. For linear arrays, 8 elements may be triggered simultaneously, while for the curved array, all elements are triggered at the same time. The whole two-dimensional sonographic image is constructed step-by-step, by stimulating one group of elements after the other over the whole array. The lines are oriented parallel to form a rectangular (e.g., corresponding to a linear array) or a divergent image (e.g., corresponding to a curved array). According to an example, a linear array may have the following dimensions: about 10-5 L and about 7-2 L-60 mm wide). Moreover, according to another example, which is in no way intended to limit the invention, a microconvex array may have the following dimensional characteristics: about 8-3 MC.

Various ultrasound probes as described herein may use 64 elements of piezoelectric array heads for micro-convex, linear and phased arrays. Moreover, the same or similar enclosure may be used for each type of probe. Linear array probes produce sound waves parallel to each other which correspond to a rectangular image. The width of the image and number of scan lines are preferably the same at all tissue levels. This has the advantage of good near field resolution. The linear array frequencies may vary from about 6 MHz to about 13 Mhz, but could be higher or lower depending on the desired embodiment. Moreover, linear arrays may incorporate any one or more of the following common applications as would be appreciated by one skilled in the art upon reading the present description:
Breast
Musculoskeletal
Nerve
Small Parts
Vascula

However, when linear arrays are applied to a curved parts of the body, they create air gaps between the skin of the patient being examined and the transducers. Accordingly, a contact head of an ultrasound probe may implement a micro-convex array producing a fan like image that is narrow near the transducers and increases in width as penetration depths are increased. Micro-convex arrays may be useful when scanning between the ribs of a patient as it may fit in the inter-costal space. However, some micro-convex arrays have poor near field resolution. An illustrative frequency range for micro-convex arrays may be from about 2.5 MHz to about 7.5 MHz, but may be higher or lower. For example, general purpose examinations may use a micro-convex probe operating at 3.5 MHz. However, for obstetric purposes either convex or linear probes may be used at an operational frequency of about 3.5 MHz. Furthermore, for pediatric applications or patients having a small and/or thin body structure, operational frequencies may be about 5 MHz. Alternatively, phased array probes may be used at operational frequencies of about 2 MHz to about 8 MHz, but could be higher or lower.

FIG. 8 illustrates an exemplary linear array contact head 800 according to one embodiment. As an option, the present head 800 may be implemented in conjunction with features from any other embodiment listed herein, such as those described with reference to the other FIGS. Of course, however, such head 800 and others presented herein may be used in various applications and/or in permutations which may or may not be specifically described in the illustrative embodiments listed herein. Further, the head 800 presented herein may be used in any desired environment. Thus FIG. 8 (and the other FIGS.) should be deemed to include any and all possible permutations.

Looking to FIG. 8, the linear array head 800 having 64 elements incorporated therewith, but may include more or fewer elements depending on the desired embodiment. Moreover, the width of the head is preferably approximately equal to the length of the array of 64 elements.

Illustrative dimensions of head 800 are also presented in FIG. 8, but are in no way intended to limit the invention. For example, a length of the head 800 may be from about 1 inches to about 3 inches, while a width of the head 800 may be from about 1 inches to about 3 inches, but may be higher or lower depending on the desired embodiment.

To form each of the elements, a piece of piezoelectric material may be cut into separate pieces called elements; each element has its own electrical circuit. These elements are arranged in a line that are fired in groups of 8 elements which preferably creates a 2-D image that consisting of parallel scan lines emitted at different points along the face of the transducer. The width of the 64-element wafer is about 2 in while the height of the 64-element wafer is about 0.5 in.

In order to achieve improved axial resolution, each element is preferably separated by about 0.05 in along the 2 in piezoelectric array. This design also preferably takes into consideration that the pulses from the 64-elements are all used to form each scan line. At each line, a different delayed pulse sequence may be used to form the unique interference pattern, resulting in a highly focused ultrasound beam perpendicular to the transducer face.

The design calls for firing 8 elements at a time because a beam produced by a narrow element will attenuate rapidly and result in lateral resolution due to beam divergence and low sensitivity due to the small element size. The transducer head may further be controlled by electronics which may be used to produce each scan line. For example, when the head is placed on the field or region of view, the firing of the inner elements may be delayed with respect to the outer elements producing a focused beam that is optimum to be processed. The time delay determines the depth of focus for the transmitted beam and can be changed during scanning. The same delay factors are also applied to the next 8 elements and then the next 8 to form the scan.

FIG. 9 shows an 8 element transducer component array 900 according to an exemplary embodiment. As an option, the present array 900 may be implemented in conjunction with features from any other embodiment listed herein, such as those described with reference to the other FIGS., such as FIG. 8. Of course, however, such array 900 and others presented herein may be used in various applications and/or in permutations which may or may not be specifically described in the illustrative embodiments listed herein. Further, the array 900 presented herein may be used in any desired environment. Thus FIG. 9 (and the other FIGS.) should be deemed to include any and all possible permutations.

Looking now to FIG. 10, the embodiment depicted therein shows a micro-convex contact head 1000 according to an exemplary embodiment. As an option, the present micro-convex head 1000 may be implemented in conjunction with features from any other embodiment listed herein, such as those described with reference to the other FIGS. Of course, however, such micro-convex head 1000 and others presented herein may be used in various applications and/or in permutations which may or may not be specifically described in the illustrative embodiments listed herein. Further, the micro-convex head 1000 presented herein may be used in any desired environment. Thus FIG. 10 (and the other FIGS.) should be deemed to include any and all possible permutations.

Illustrative dimensions of head 1000 are also presented in FIG. 10, but are in no way intended to limit the invention. For example, a length of the head 1000 may be from about 1 inches to about 3 inches, while a width of the head 1000 may be from about 1 inches to about 3 inches, but may be higher or lower depending on the desired embodiment. Moreover, as described above with reference to FIG. 8, the number of elements and/or wires coupled thereto is preferably not limited to 64 as denoted in the illustration of the present embodiment.

Referring still to FIG. 10, the micro-convex head 1000 may have an operational frequency in a range from about 2 MHx to about 8 MHz. Moreover, the contact area of the micro-convex head 1000 is preferably curved, e.g., so to have a smaller contact surface, which improves the coupling between the transducers and the skin surface of a patient, even in complicated areas such as the supraclavicular or jugular fossa.

Various micro-convex heads having large aperture and selection of transmission frequencies may also be used in gynecological diagnostic. In such applications, the width of the 64-elements may be about 1 wavelength each. Moreover, preferably all the elements are arranged in an arc shape. Furthermore, for embodiments having arc shaped element arrays, it is preferred that not all elements are fired at the same time. Rather, the embodiment may fire 8 elements at a time. As a result, the array may have a wide and/or far field of view. Moreover, such embodiments may preferably have an operational frequency from about 2 MHz to about 8 MHz.

FIG. 11 illustrates a phased transducer array contact head 1100 according to an exemplary embodiment. As an option, the present head 1100 may be implemented in conjunction with features from any other embodiment listed herein, such as those described with reference to the other FIGS. Of course, however, such head 1100 and others presented herein may be used in various applications and/or in permutations which may or may not be specifically described in the illustrative embodiments listed herein. Further, the head 1100 presented herein may be used in any desired environment. Thus FIG. 11 (and the other FIGS.) should be deemed to include any and all possible permutations.

Illustrative dimensions of head 1100 are also presented in FIG. 1I, but are in no way intended to limit the invention. For example, a length of the head 1100 may be from about 1 inches to about 3 inches, while a width of the head 1100 may be from about 1 inches to about 3 inches, but may be higher or lower depending on the desired embodiment. Moreover, as described above with reference to FIG. 8, the number of elements and/or wires coupled thereto is preferably not limited to 64 as denoted in the illustration of the present embodiment.

Referring still to FIG. 11, the phased array transducer head 1100 preferably has 64 elements that are arranged in matrix fashion. The width of each transducer element may be from about to about 2 of an operational frequency wavelength, as described below. The crystals are preferably pulsed almost simultaneously to produce an image scan line. An illustrative range of operational frequencies for the head 1100 may be from about 2 MHz to about 8 MHz, but may be higher or lower depending on the desired embodiment.

Multiple, miniscule pulses steer & focus the beam(s) emitted from the head 1100 into a sector-shaped image by varying the time delay minutely in the pulsing sequence of the elements. They may be electronically-focused & steered along the sound path, mechanically, e.g., focused along an elevational axis. It follows that each of the elements are preferably fired simultaneously. An exemplary advantage of using phased array is that it has a small footprint for tight acoustic windows.

Head 1100 and/or any other embodiment herein may be implemented in combination with an ultrasound probe having artificial intelligence software that preferably aids a user (e.g., ultrasound technician) in selecting the contact head configuration for the type of the examination that needs to be conducted on a patient (e.g., humans and animals). When the user activates the functional button (e.g., as described above), the ultrasound probe may guide the user while examining a patient, e.g., noting how much pressure should be applied, a desired angle of contact, adjustments to the gain, brightness, power, depth, auto adjusts to the resolution of the image and/or the cine loop, etc. Moreover, a screen of a graphical user interface may display an output of the ultrasound probe, while also indicating when the probe is positioned at a desired location. Accordingly, the artificial intelligence software may automatically freeze the display produced on the screen of a graphical user interface while additional changes may be made to the zoom of the image and/or the patient's EMR.

As described above, various embodiments herein may implement flexible PCBs. By routing the signals of each individual transducer element through the multiple PCB, cost of the embodiments described herein has been greatly reduced from those associated with traditional products. Additional advantages of using flexible PCBs may include any of the following, depending on the embodiment:
Flexible PCBs are able to fit in tight spaces. They can bend, fold, twist, change in width many times and even flex from a rolled configuration. This gives the package designer the freedom to relocate other parts and subassemblies where they will optimize circuit and equipment operation. The designer is no longer restricted by the space demands of bulky, rigid PC boards
Simplifying circuit geometry and placing surface mount devices directly on the circuit can also improve the circuit design. Intricate patterns that may be difficult to achieve with rigid board connector pins can be designed into the flexible circuit artwork. Greater circuit complexity is achieved in a much smaller space
Because flexible circuits can be bent, twisted and rolled to suit the contour of the equipment, designers can enjoy a space savings of up to 75%.
Elimination of mechanical connectors.
Unparalleled design flexibility.
Size and weight reduction.

Ultrasound probes may include three 2 in ×3 in and one 1 in ×2 in double sided surface mountable PCBs. A first of the PCBs may be used to embed a waveform generator, high voltage (+/−100V) power circuits, high voltage amplifiers, multiplexer, T/R switch, etc. The first of the PCBs also preferably has electromagnetic interference isolation (EMI).

A second of the PCBs may be used for the receive portion of the probe, e.g., having the same or similar dimensions as a transmit PCB. It is also double sided and surface mounts 16 channel low noise amplifier (LNA), variable gain control (VGA) circuitry, anti-aliasing filter (AAF), mixers and/or analog to digital convertors (ADCs) that may be encapsulated in a low power compact package and has full EMI isolation from other components.

A third of the PCBs may be a high compute intensive PCB which houses processors and memory to compute transmit and receive beams, software to enhance the human machine interaction by embedding intelligent HMI functionality. Echo analytics and with graphical descriptive, high resolution image processing; high speed connectivity and high performance 512×512 raster generation. This third PCB may further have fast access flash and RAM to carry out the above functions with virtually no delay.

The fourth PCB (e.g., the 1 in ×2 in PCB) preferably incorporates an IEEE 802.11n/ac module which uses ultra-low-power components having dual bands for WiFi which may operate at rates of up to about 433 Mbps. It may further support MIMO, e.g., for packet reception and beam forming feedback, for enhanced coexistence and network throughput in any 802.11 ac network(s). The PCB may further include an in-built antenna that may have a range of about 20 meters. Moreover, a processor with on chip memory may achieve high-throughputs and may further enable processing Wi-Fi security and/or provide the functionality desired to achieve a robust transmission.

As described above, various ultrasound probes included in the various embodiments herein preferably include a battery. FIG. 12 depicts a battery compartment 1200, in accordance with one embodiment. As an option, the present battery compartment 1200 may be implemented in conjunction with features from any other embodiment listed herein, such as those described with reference to the other FIGS. Of course, however, such battery compartment 1200 and others presented herein may be used in various applications and/or in permutations which may or may not be specifically described in the illustrative embodiments listed herein.

Further, the battery compartment 1200 presented herein may be used in any desired environment. Thus FIG. 12 (and the other FIGS.) should be deemed to include any and all possible permutations. Moreover, it should be noted that exemplary dimensions are presented with the battery compartment 1200 of FIG. 12 which are in no way intended to limit the various configurations the battery compartment 1200 may have, e.g., according to a desired embodiment.

Illustrative dimensions of battery compartment 1200 are also presented in FIG. 12, but are in no way intended to limit the invention. For example, a length of the battery compartment 1200 may be from about 2 inches to about 4 inches, while a width of the battery compartment 1200 may be from about 1 inches to about 3 inches, but may be higher or lower depending on the desired embodiment.

Referring still to FIG. 12, the battery compartment 1200 is preferably located at a bottom side 1204 of an ultrasound probe according to any of the embodiments herein. Moreover, the battery compartment 1200 preferably houses a battery 1202 having a positive electrode 1212. In a preferred approach, the battery 1202 includes a 3000 mAH Lithium-Ion rechargeable battery. Moreover, the battery 1202 may further have continuous operation capacity of about one hour. In preferred approaches, the battery 1202 may be about 2.6 in ×0.7 in, and cylindrical in shape.

Additionally, the battery compartment 1200 includes latches 1206 of a battery holder 1208 in addition to screw threads 1210, e.g., for coupling the battery compartment 1200 to an ultrasonic probe according to any of the embodiments herein.

The inventive concepts disclosed herein have been presented by way of example to illustrate the myriad features thereof in a plurality of illustrative scenarios, embodiments, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various embodiments have been described above, it should be understood that they have been presented by way of example only, and not limitation. Thus, the breadth and scope of an embodiment of the present invention should not be limited by any of the above-described exemplary embodiments, but should be defined only in accordance with the following claims and their equivalents.

What is claimed is:

1. An ultrasound probe, comprising:
a housing configured for grasping by a human hand;
an array of transducers for transducing sound waves into electrical signals;
a circuit board in the housing, the circuit board having a plurality of leads, each of the transducers being coupled to at least an associated one of the leads;
processing circuitry in the housing and coupled to the circuit board for processing the electrical signals, or derivatives of the electrical signals, into sonogram data; and
an output device for outputting the sonogram data.

2. The ultrasound probe as recited in claim 1, wherein the transducers include piezoelectric devices.

3. The ultrasound probe as recited in claim 1, wherein the output device includes a wireless transmitter.

4. The ultrasound probe as recited in claim 1, wherein the output device includes a universal serial bus interface compatible with the USB 3.0 standard.

5. The ultrasound probe as recited in claim 1, wherein the output device includes an ethernet interface.

6. The ultrasound probe as recited in claim 1, wherein the circuit board is flexible.

7. The ultrasound probe as recited in claim 1, wherein an average outer diameter of the housing along a longitudinal axis thereof is less than 2 inches.

8. The ultrasound probe as recited in claim 1, comprising a battery for powering the processing circuitry.

9. The ultrasound probe as recited in claim 1, comprising a heat sensor coupled to the housing.

10. The ultrasound probe as recited in claim 1, comprising a light source for illuminating an environment near the housing.

11. The ultrasound probe as recited in claim 1, comprising a control on the housing for adjusting at least one of a frequency and depth of the sound waves.

12. The ultrasound probe as recited in claim 1, comprising a detachable contact head selected from a group consisting of a linear array configuration, a micro-convex configuration and a phased array configuration.

13. The ultrasound probe as recited in claim 1, comprising a control for selecting an operational mode selected from a group consisting of a linear array mode, a micro-convex mode and a phased array mode.

14. An ultrasound probe, comprising:
a housing configured for grasping by a human hand;
an array of piezoelectric transducers for generating sound waves and for transducing reflected ones of the sound waves into electrical signals;
a flexible circuit board in the housing, the circuit board having a plurality of leads, each of the transducers being coupled to at least an associated one of the leads;
processing circuitry in the housing and coupled to the circuit board for processing the electrical signals, or derivatives of the electrical signals, into sonogram data;
an output device for outputting the sonogram data, wherein the output device includes a wireless transmitter;
a battery for powering the processing circuitry; and
a control on the housing for adjusting at least one of a frequency and depth of the sound waves.

15. The ultrasound probe as recited in claim 14, wherein the output device further includes a universal serial bus interface compatible with the USB 3.0 standard.

16. The ultrasound probe as recited in claim 14, wherein the output device further includes an ethernet interface.

17. The ultrasound probe as recited in claim 14, comprising a heat sensor coupled to the housing.

18. The ultrasound probe as recited in claim 14, comprising a light source for illuminating an environment near the housing.

19. The ultrasound probe as recited in claim 14, comprising a detachable contact head selected from a group consisting of a linear array configuration, a micro-convex configuration and a phased array configuration.

20. The ultrasound probe as recited in claim 14, comprising a control for selecting an operational mode selected from a group consisting of a linear array mode, a micro-convex mode and a phased array mode.
